(19) United States
(12) Patent Application Publication
(10) Pub. No.: US 2022/0335769 A1
(43) Pub. Date: Oct. 20, 2022

(54) APPARATUS FOR HANDLING NOTES OF VALUE

(71) Applicant: Wincor Nixdorf International GmbH, Paderborn (DE)

(72) Inventors: Richard Dusterhus, Paderborn (DE); Gerhard Schniedermann, Paderborn (DE)

(21) Appl. No.: 17/762,040

(22) PCT Filed: Sep. 24, 2020
(86) PCT No.: PCT/EP2020/076657

§ 371 Date: Mar. 18, 2022

(30) Foreign Application Priority Data
Sep. 26, 2019 (DE) 10 2019 125 944.0

(51) Int. Cl. G07D 11/14; G07D 11/22; G07D 11/13

(52) U.S. Cl. CPC G07D 11/14; G07D 11/22; G07D 11/13; G07D 2211/00

(57) ABSTRACT

An apparatus for handling notes of value can comprise a dispensing compartment for withdrawing the notes of value. The dispensing compartment is bounded on a first side by a depositing element and on a second side by a delimiting element. Furthermore, the apparatus comprises at least one first sensor unit for detecting an initialization position of the depositing element. The first sensor unit is designed to generate a first sensor signal and to transmit said first sensor signal to a control unit. The control unit is designed to take the first sensor signal as a basis for actuating a first drive unit such that the depositing element is moved a predetermined distance away from the delimiting element from the initialization position to a first supply position for supplying the notes of value.

The invention relates to a device for handling bills, said device comprising a dispensing compartment for retrieving bills. The dispensing compartment on a first side is delimited by a depositing element, and on a second side is delimited by a delimiting element. The device furthermore comprises a control unit.

Devices for handling bills, in particular automatic teller machines, in which the bills are offered to a user as a wad are known from the prior art. For this purpose, the bills are retrieved from bill cassettes, fed to an intermediate storage, and by a stacking unit stacked so as to form a bill stack which is then transported to the dispensing compartment with the aid of a transport unit, for example with the aid of a displacement slide.

In these devices, it is disadvantageous that a lot of installation space is required for the intermediate storage, the stacking unit and the transport unit, and that the intermediate storage in particular slows down the disbursement procedure.

It is an object of the invention to specify a device for handling bills with the aid of which bills can be provided for dispensing in a simple manner.

This object is achieved by a device having the features of claim 1. Advantageous refinements are set forth in the dependent claims.

The device having the features of claim 1 comprises at least one first sensor unit for detecting an initializing position of the depositing element. The first sensor unit is configured to generate a first sensor signal and to transmit the latter to the control unit. The control unit is configured to actuate a first drive unit for moving the depositing element, wherein the control unit, proceeding from the first sensor signal, actuates the first drive unit in such a manner that the depositing element is moved by a predetermined distance away from the delimiting element, from the initializing position to a first feeding position for feeding the bills. Particularly simple and reliable controlling of the movement of the depositing element to the feeding position is achieved as a result.

It is particularly advantageous for the first sensor unit to comprise at least one sensor element, wherein the first sensor unit is disposed in such a manner that a first position of the sensor element is able to be detected with the aid of the first sensor unit, and for the first sensor unit to be configured to emit the first sensor signal as a function of the detected first position of the sensor element. Particularly reliable monitoring of the position of the sensor element is achieved as a result.

It is particularly advantageous for the first sensor unit to detect the first position of the sensor element when the depositing element is situated in the initializing position, wherein the sensor element contacts the depositing element and as a result of contacting the depositing element is moved in the direction of the delimiting element. It is achieved as a result that the initializing position is determined in a simple and accurate manner.

In one particularly advantageously embodiment, the sensor element is a contact pressure element which for the orderly depositing of the bills is pivotably connected to the delimiting element, wherein the sensor element is movable between the raised first position and a lowered second position. An uncontrolled movement of the bills during feeding into the dispensing compartment is prevented as a result. As a result, an orderly bill stack is formed in the dispensing compartment.

It is advantageous for the device to comprise at least one second sensor unit for detecting a lower position of the depositing element, wherein the second sensor unit is configured to generate a second sensor signal and to transmit the latter to the control unit. Furthermore, the control unit is configured to actuate the first drive unit for moving the depositing element as a function of the second sensor signal. Simple and reliable controlling of the movement of the depositing element is achieved as a result.

It is furthermore advantageous for the control unit, proceeding from the second sensor signal, to be configured to actuate the first drive unit in such a manner that the depositing element is moved by a predetermined distance toward the delimiting element, to the initializing position. A secure movement of the depositing element to the initializing position is achieved as a result.

It is particularly advantageous for the device to comprise a closure element which in a closed position delimits the dispensing compartment on a third side, and which in an opened position enables access to bills provided in the dispensing compartment, wherein the closure element for opening the dispensing compartment is movable from the closed position to an opened position, and for closing the dispensing compartment is movable from the opened position to the closed position. Easy access to the bills provided for disbursement is enabled as a result.

In one advantageous embodiment, the device comprises at least one third sensor unit for detecting the closed position and/or at least one fourth sensor unit for detecting the opened position of the closure element. The third sensor unit is configured to generate a third sensor signal and to transmit the latter to the control unit, and/or the fourth sensor unit is configured to generate a fourth sensor signal and to transmit the latter to the control unit. The control unit is configured to actuate a second drive unit for driving the closure element as a function of the third sensor signal and/or of the fourth sensor signal. Automated and simple controlling of the movement of the closure element is achieved as a result.

It is advantageous for the device to comprise a fifth sensor unit which is in particular disposed in the dispensing compartment and which is configured to generate a fifth sensor signal and to transmit the latter to the control unit when at least one bill is received in the dispensing compartment. Simple monitoring of the dispensing compartment is achieved as a result.

It is furthermore advantageous for a plurality of bills received in the dispensing compartment to form a bill stack, wherein the bills of the bill stack are disposed on top of one another in such a manner that the lowermost bill by way of the front side thereof or the rear side thereof bears on the depositing element and that the uppermost bill by way of the front side thereof or the rear side thereof contacts the sensor element, wherein the direction from the lowermost bill to the uppermost bill is a stacking direction of the bill stack. Secure and reliable depositing and stacking of the bills is possible as a result.

In one particularly advantageous embodiment, the first sensor unit detects the first sensor element position when the bill stack contacts the sensor element, wherein the sensor element as a result of contacting the bill stack is moved in the direction of the delimiting element. Simple monitoring of the height of the bill stack is achieved as a result.

It is advantageous for the control unit, proceeding from the reception of the first sensor signal and of the fifth sensor signal, to actuate the first drive unit in such a manner that the depositing element is moved by a predetermined distance away from the delimiting element, from the first feeding position to a second feeding position, or from the second feeding position to a third feeding position, in such a manner that further bills are able to be fed into the dispensing compartment. Orderly depositing and stacking of the bills in the dispensing compartment is ensured as a result.

It is particularly advantageous for the control unit for opening the dispensing compartment to be configured to actuate the first drive unit in such a manner that the depositing element, proceeding from the lower position or from the first feeding position or from the second feeding position or from the third feeding position, is moved by a predetermined distance to an entrainment position, wherein the delimiting element in the movement of the depositing element is moved from a first delimiting position to a second delimiting position, wherein an engagement element of the delimiting element comes to engage with an engagement region of the closure element. As a result, it is achieved that the volume of the dispensing compartment when opening is enlarged in a particularly simple manner such that particularly easy access is possible to the bills provided for disbursement.

It is advantageous for the first sensor unit and/or the second sensor unit and/or the third sensor unit and/or the fourth sensor unit and/or the fifth sensor unit to comprise at least one detection element, wherein the detection element generates the sensor signal and transmits the latter to the control unit, and wherein the detection element preferably comprises a light barrier. As a result, it is achieved that the sensor units are of a simple construction and cost-effective.

It is furthermore advantageous for the device to comprise at least one third drive unit for driving transport elements for transporting the bills, wherein the transport elements for feeding the bills into the dispensing compartment are drivable in a first direction, and wherein the transport elements for transporting the bills out of the dispensing compartment in a direction of a bill handling unit of the device are drivable in a second direction, counter to the first direction. A compact construction mode of the device is achieved as a result.

It is particularly advantageous for the device to comprise a sixth sensor unit for detecting at least one feature of the bills to be disbursed, wherein the control unit as a function of the feature detected by the sixth sensor unit is configured to distinguish whether the bill is a bill cleared for disbursement, and for the control unit to drive the third drive unit in the first direction when the sixth sensor unit detects a bill cleared for disbursement. It is achieved as a result that bills which are cleared for disbursement exclusively are fed to the dispensing compartment.

In one advantageous embodiment, the control unit is configured to actuate the second drive unit in such a manner that the closure element for retrieving the bills is disposed in the opened position for a predetermined period, and after the pre-set period is moved to the closed position. Furthermore, the control unit, after the pre-set period has elapsed, is configured to check whether the fifth sensor unit relays the fifth sensor signal to the control unit. Should the check result in the fifth sensor signal being relayed to the control unit, the control unit is configured to actuate the first drive unit in such a manner that the depositing element is moved from the entrainment position to a discharge position and to actuate the third drive unit in such a manner that the transport elements are driven in the second direction. It is achieved as a result that bills which are not retrieved from the dispensing compartment as so-called retracts are securely transported out of the dispensing compartment, preferably into a retract-reject container.

Further features and advantages are derived from the description hereunder which in conjunction with the appended figures explains in more detail exemplary embodiments.

In the figures:

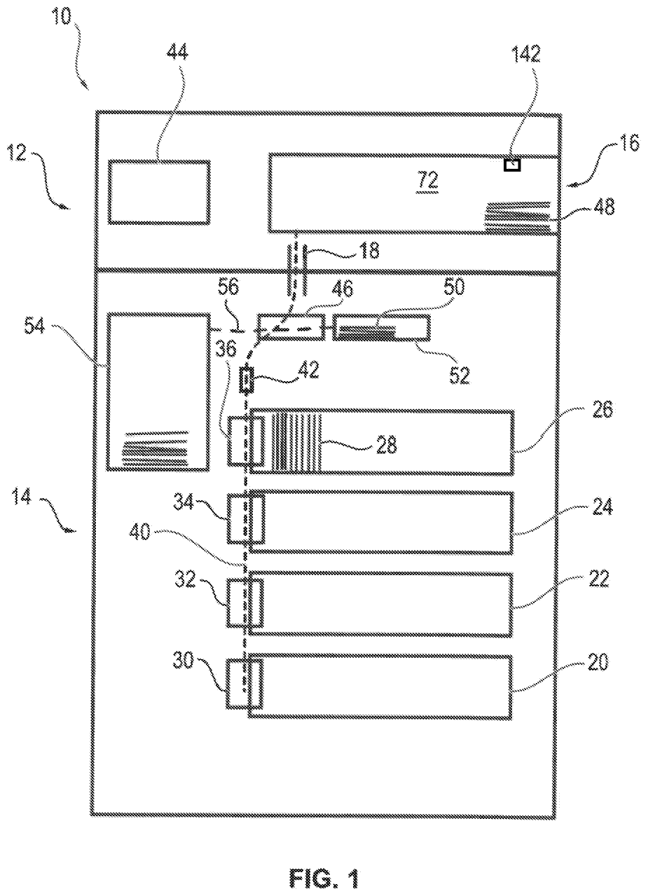

FIG. 1 shows a schematic illustration of a device for handling bills;

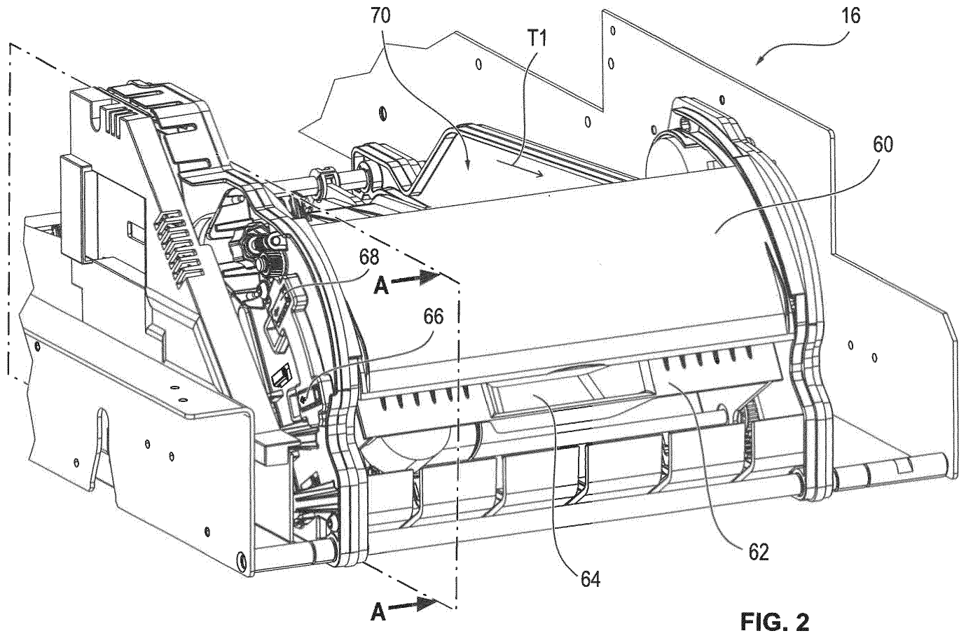

FIG. 2 shows a perspective detailed view of a bill dispensing unit according to a first embodiment, having a closed closure element for use in the device for handling bills as per FIG. 1;

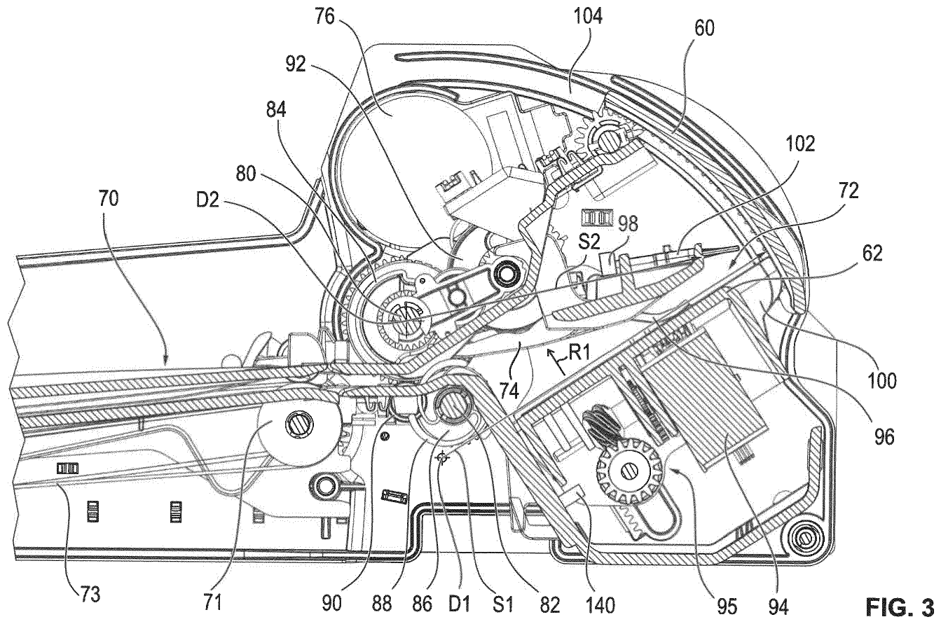

FIG. 3 shows a sectional illustration of a fragment of the bill dispensing unit as per FIG. 2;

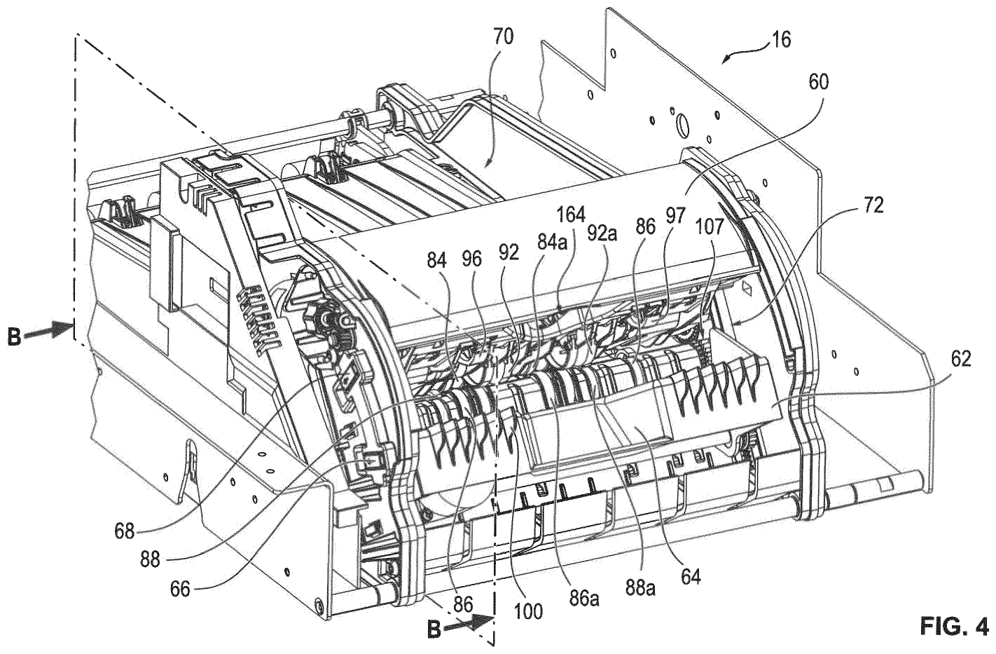

FIG. 4 shows a perspective detailed view of the bill dispensing unit as per FIGS. 2 and 3, having an opened closure element;

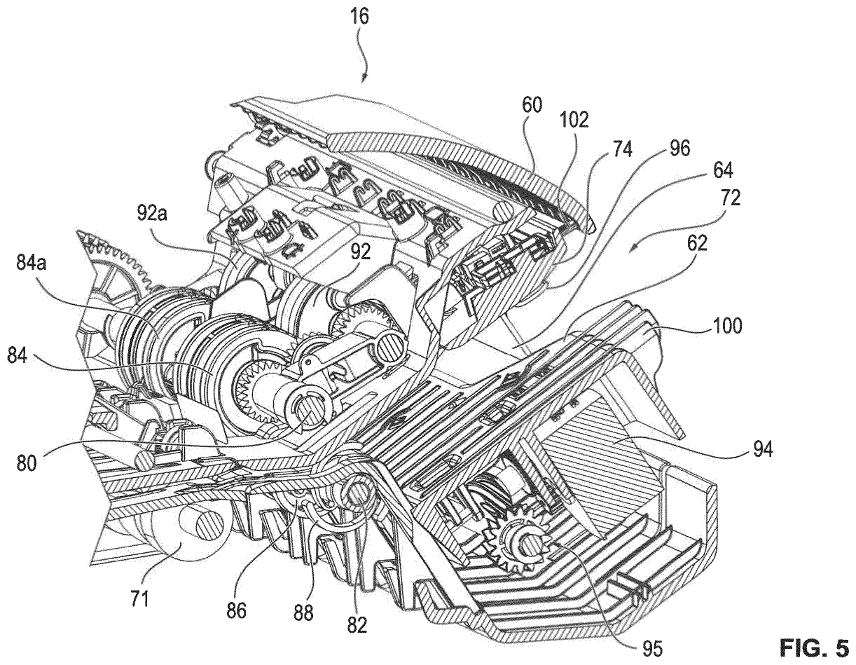

FIG. 5 shows a fragment of a perspective sectional illustration of the bill dispensing unit as per FIG. 4;

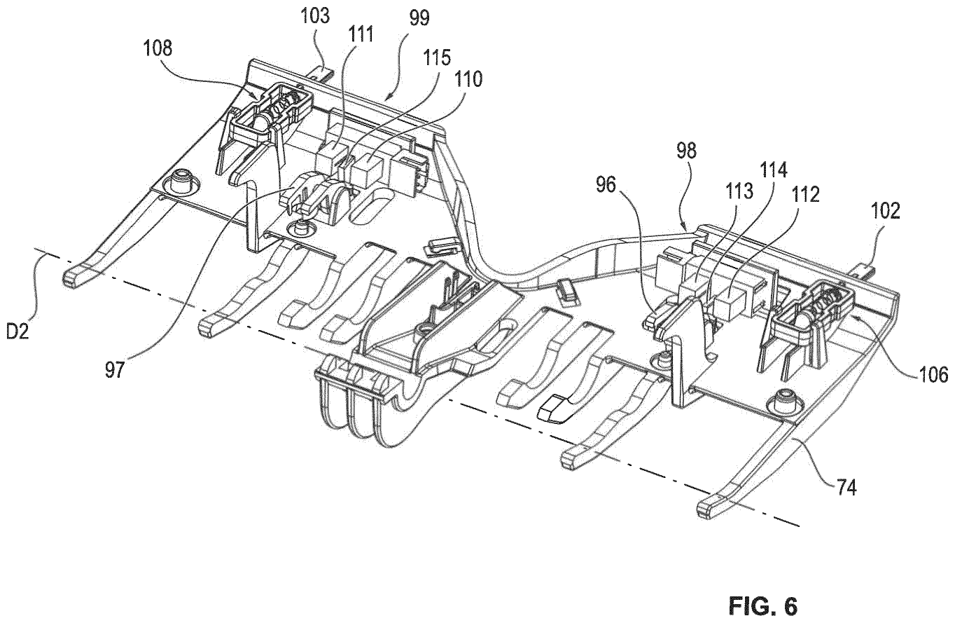

FIG. 6 shows a perspective view of a delimiting element of the bill dispensing unit as per FIGS. 2 to 5;

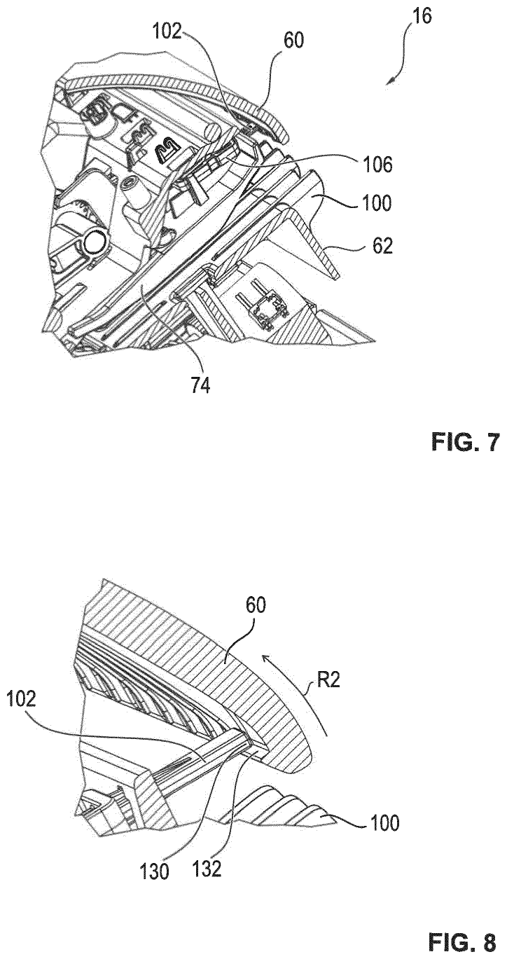

FIG. 7 shows a perspective sectional illustration of several elements of the bill dispensing unit as per FIGS. 2 to 6;

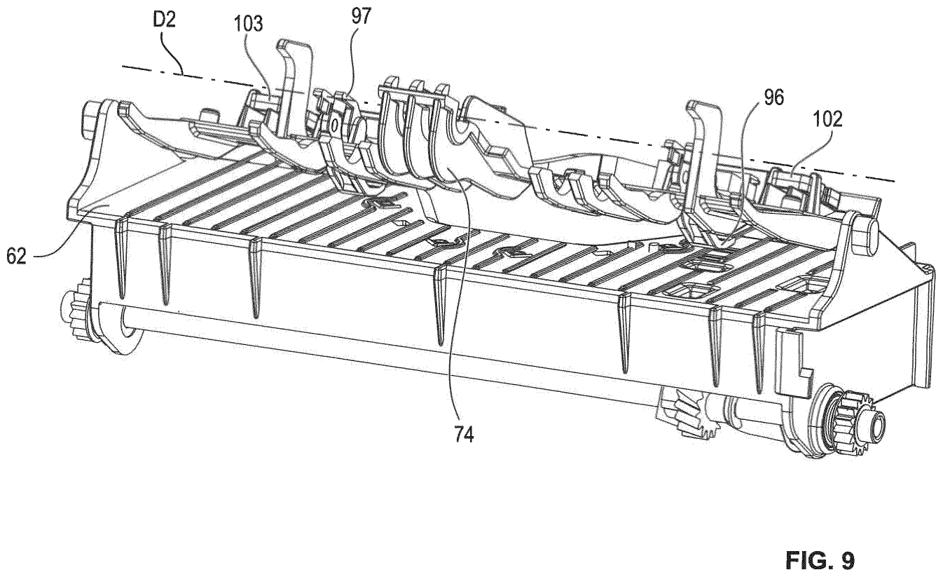

FIG. 8 shows an enlarged detailed view of an engagement element as per FIG. 7;

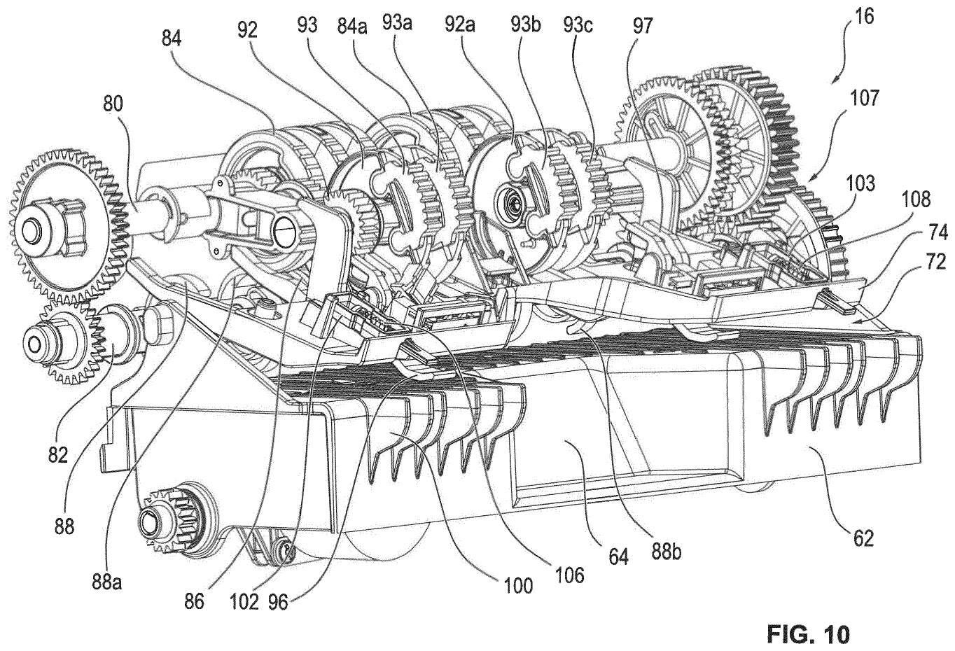

FIG. 9 shows a perspective detailed view of a depositing element and of the delimiting element of the bill dispensing unit as per FIGS. 2 to 8;

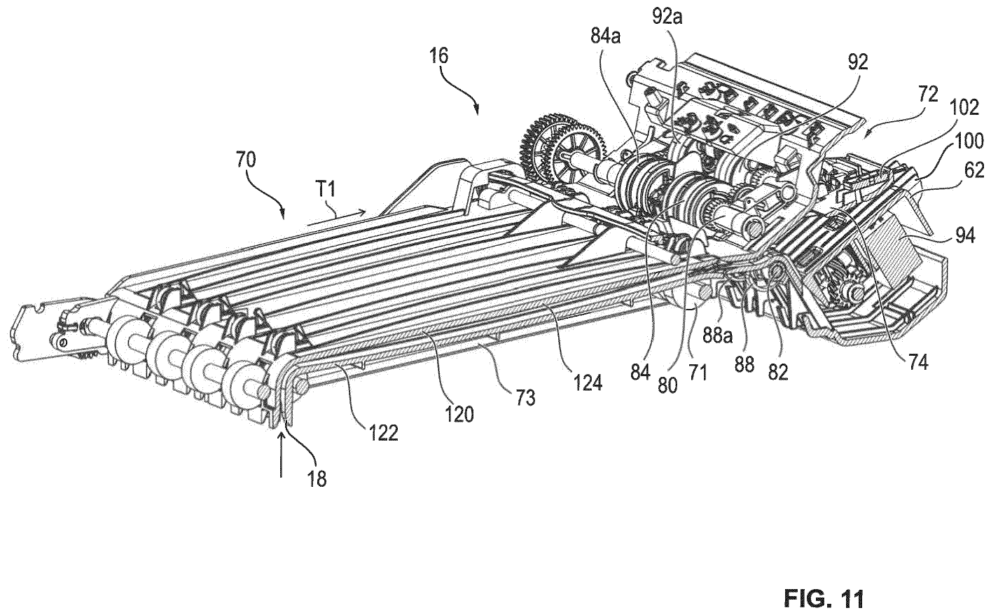

FIG. 10 shows a further perspective detailed view of the bill dispensing unit as per FIGS. 2 to 9;

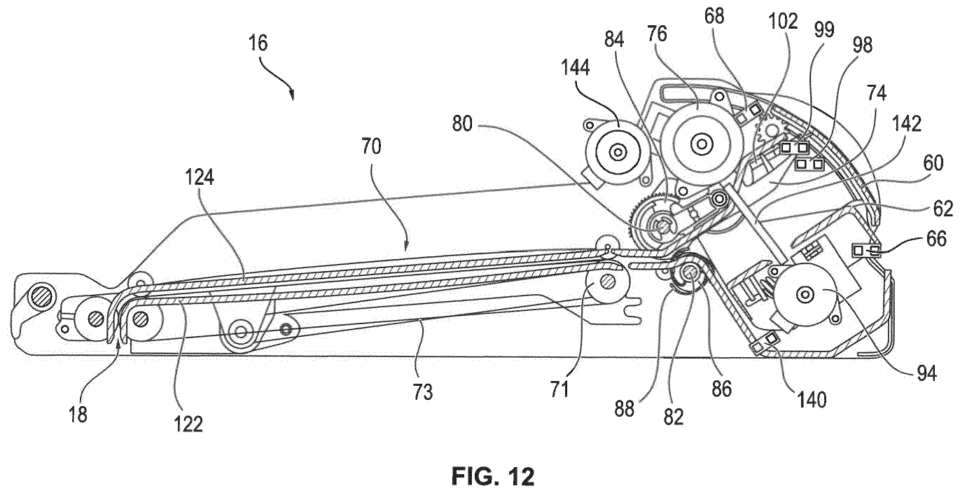

FIG. 11 shows a further perspective detailed view of the bill dispensing unit as per FIGS. 2 to 10;

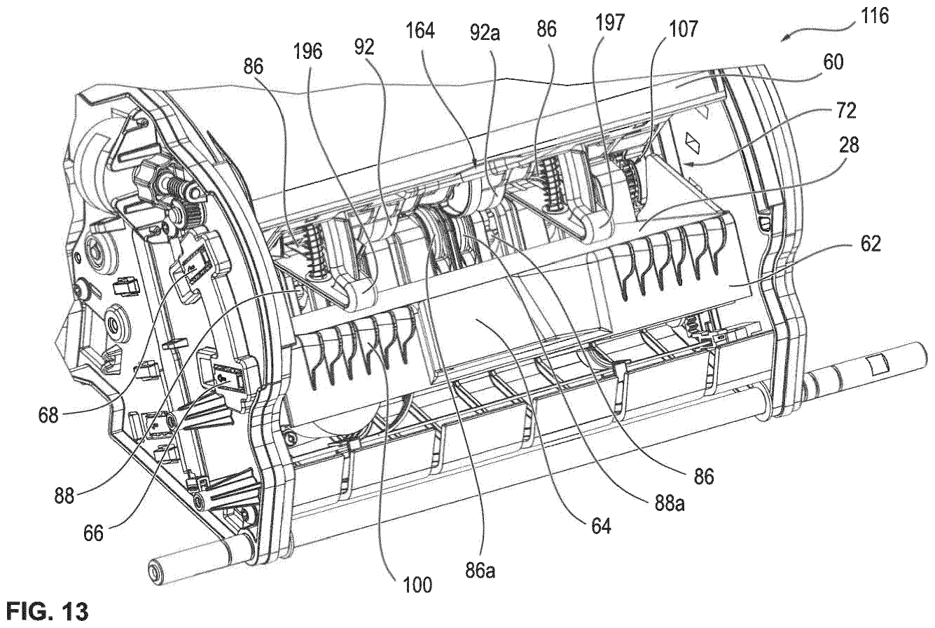

FIG. 12 shows a further schematic illustration of the bill dispensing unit as per FIGS. 2 to 11;

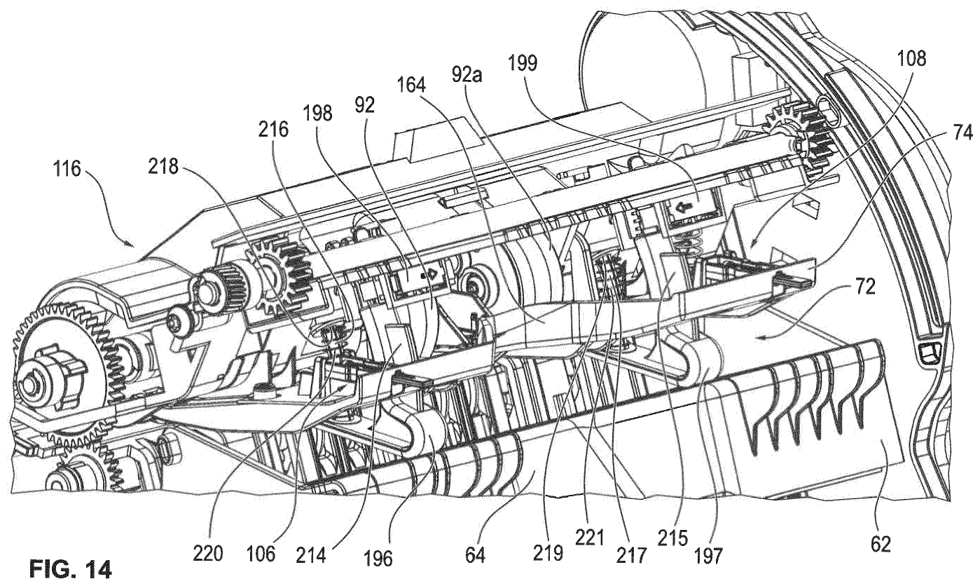

FIG. 13 shows a perspective view of a bill dispensing unit according to a second embodiment which, as an alternative to the bill dispensing unit as per FIGS. 2 to 12, is suitable for use in the device as per FIG. 1;

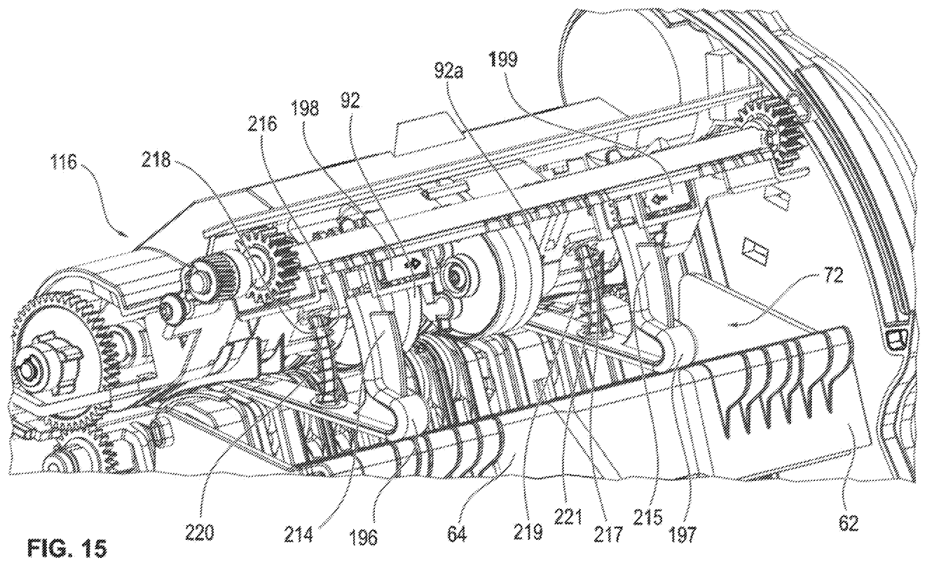

FIG. 14 shows a perspective detailed view of a fragment of the bill dispensing unit as per FIG. 13;

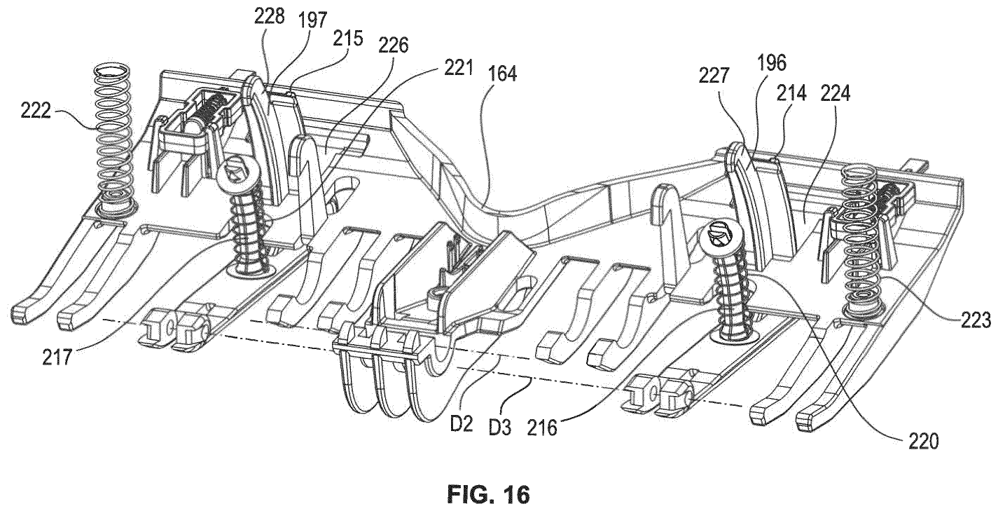

FIG. 15 shows the fragment of the bill dispensing unit as per FIG. 14, wherein a delimiting element of the bill dispensing unit has been omitted;

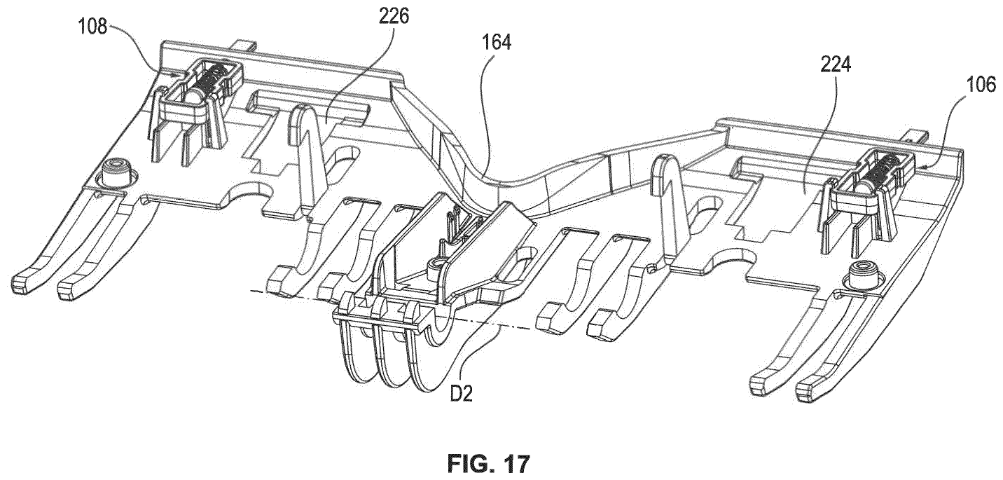

FIG. 16 shows a perspective detailed view of the depositing element and of contact pressure elements of the bill dispensing unit as per FIGS. 13 to 15;

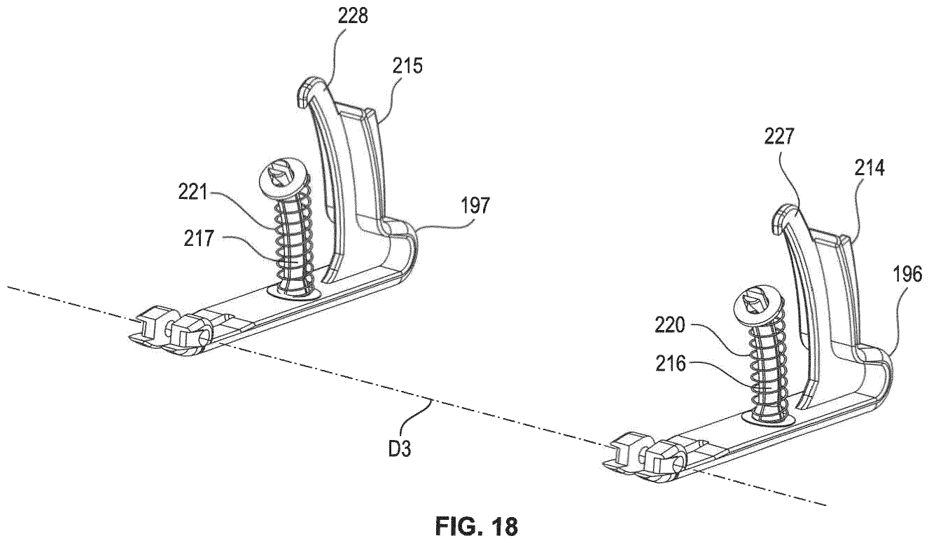

FIG. 17 shows a further perspective detailed view of the dispensing element as per FIG. 16, without contact pressure elements;

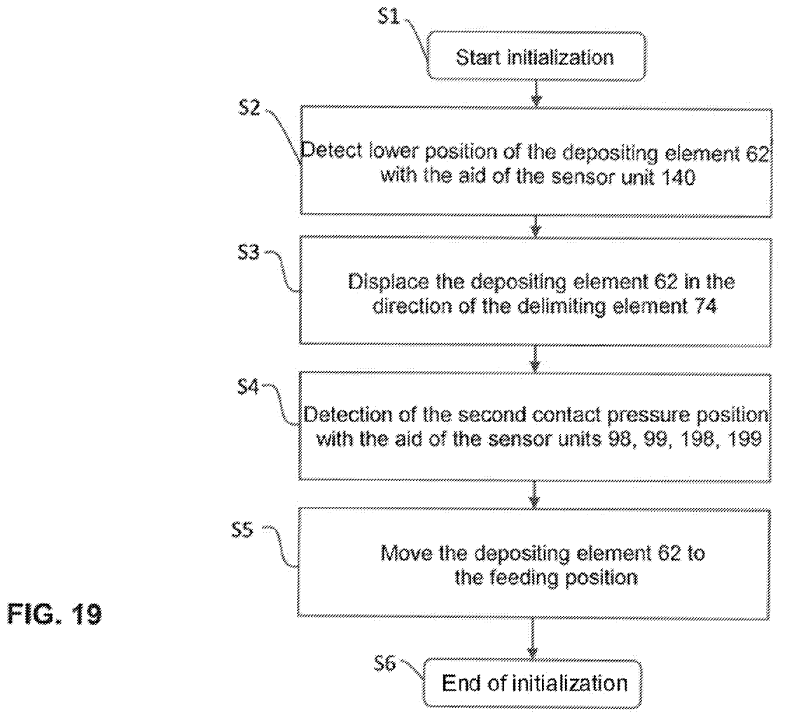

FIG. 18 shows a further perspective detailed view of the contact pressure elements as per FIG. 16;

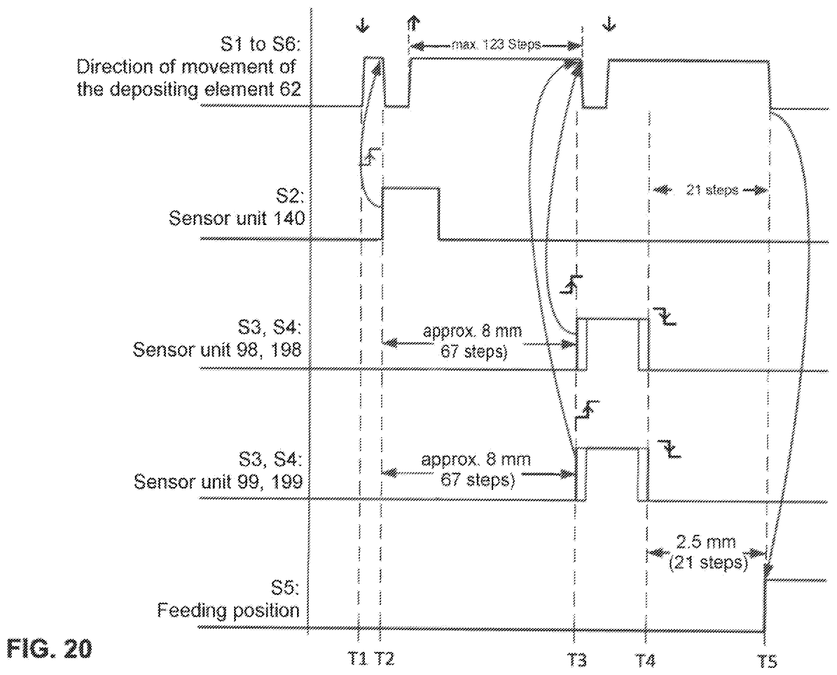

FIG. 19 shows a flow chart of a method for initializing a bill dispensing unit as per one of FIGS. 2 to 18; and

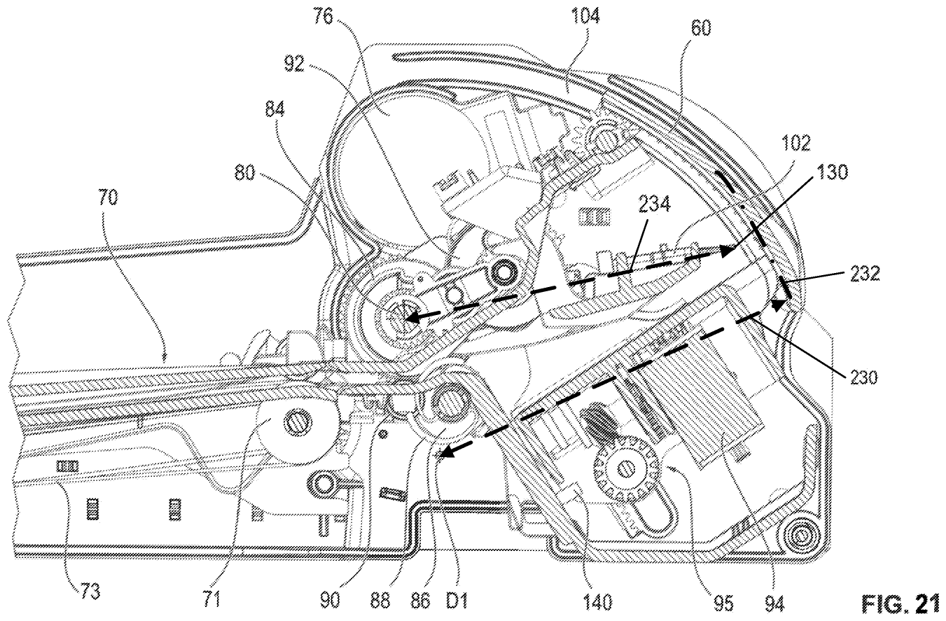

FIG. 20 shows a signal diagram for controlling the initialization according to the sequence as per FIG. 19.

FIG. 1 shows a schematic illustration of a device 10 for handling bills 28, 48, 50. The device 10 comprises a head module 12 and a strongbox module 14. The head module 12 comprises a bill dispensing unit 16 for dispensing bills 48 to be disbursed to an operator. Bills 28, 48 are transported from the strongbox module 14 into the head module 12 by way of a transfer opening 18. The strongbox module 14 comprises four bill cassettes 20 to 26 which serve for storing and for transporting bills 28.

The bills 28 are deposited in the form of a stack in the bill cassettes 20 to 26. One of these stacks is indicated in an exemplary manner in the first bill cassette 26. In an exemplary manner, one of the bills of this bill stack is identified by the reference sign 28. In the operating position of the bill cassettes 20 to 26 shown in FIG. 1, the bills 28 are disposed in the bill cassettes 20 to 26 so as to be upright on one of the edges of said bills 28. Each bill cassette 20 to 26 has in each case one opening, not illustrated, for retrieving bills 28. A singularizing unit 30 to 36, with the aid of which the bills 28 stored in the bill cassettes 20 to 26 can be retrieved and singularized as a result, is disposed in front of the opening.

As soon as a user initiates a disbursement procedure, bills 28 to be disbursed are retrieved from the corresponding bill cassettes 20 to 26 and, after singularizing, are fed to a transport path 40. A sensor unit 42 is disposed along the transport path 40 and detects at least one feature of each bill 28 to be disbursed. The sensor unit 42 transmits a measured value of the detected feature to a control unit 44 which, as a function of the detected feature, is configured to evaluate whether the bill 28 is a first bill 48 cleared for disbursement or a second bill 50 not cleared for disbursement.

The control unit 44 actuates a turnout unit 46 in such a manner that the bill 48 is fed to the bill dispensing unit 16 when said bill 48 is a bill 48 cleared for disbursement. However, if the bill is a bill 50 not cleared for disbursement, the control unit 44 actuates the turnout unit 46 in such a manner that the bill 50 is fed to an intermediate storage 52.

In order not to unnecessarily delay the disbursement procedure for the operator, replacement bills for the bills 50 not cleared for disbursement are retrieved from the bill cassettes 20 to 26 and, to the extent that said replacement bills by virtue of the features detected with the aid of the sensor unit 42 are cleared for disbursement, are transported to the bill dispensing unit 16. In this way, the desired total value can be dispensed to the operator without interrupting the disbursement procedure. Upon completion of the disbursement procedure, the bills 50 not cleared for disbursement, by way of a further transport path 56, are fed to a container 54, in particular to a so-called reject and retract container 54 which is preferably configured in the form of a further bill cassette.

The reject and retract container 54 comprises in particular a first compartment for receiving the rejected bills 50 (so-called rejects) and a second compartment, separate from the first compartment, for receiving bills 48 not retrieved from the dispensing compartment (so-called retracts). In this way, the rejects and the retracts can be separately stored, as a result of which tracing the notes is easily possible.

In an alternative embodiment, the device 10 can be configured as an automatic recycling cash machine, i.e. as a device 10 for disbursing and depositing bills. In this embodiment, the sensor unit 42, or a further sensor unit which is in particular disposed in the head module, also checks the features of the bills 28 which by way of a bill dispensing and depositing unit are fed to the device 10 by an operator. Bills 28 cleared for depositing are fed to the bill cassettes 20 to 26, while bills not cleared for depositing are fed to the intermediate storage 52. The device 10 in other embodiments can comprise a larger or smaller number of bill cassettes 20 to 26, as well as separate reject and retract containers 54.

FIG. 2 shows a perspective detailed view of the bill dispensing unit 16 of the device 10, having a closed closure element 60. Elements of identical construction or identical function have the same reference signs. The closure element 60 delimits a dispensing compartment which in the illustration of FIG. 2 is obscured by the closure element 60 and in which the bills 48 destined for disbursement are provided for retrieval by an operator. The dispensing compartment is identified in FIG. 3 and will be described in more detail hereunder. In the closed position of the closure element 60 illustrated in FIG. 3, no access to the dispensing compartment 72 and to bills 48 provided therein is possible.

A depositing element 62 for depositing the bills 48 provided for disbursement can be seen in the illustration as per FIG. 2. The depositing element 62 comprises a clearance 64 which is disposed in such a manner that the bills 48 deposited in the dispensing compartment 72 lie above a sub-region of the clearance 64. In the opened position of the closure element 60, access to the bills 48 is facilitated in that an operator can reach into the clearance 64 and below the bills 48 with his/her fingers.

Further to be seen in FIG. 2 are two sensor units 66, 68, wherein the sensor unit 66 is provided for detecting the closed position of the closure element 60, and the sensor unit 68 is provided for detecting an opened position of the closure element 60. The sensor units 66, 68 when detecting the respective position of the closure element 60 generate a sensor signal and transmit this sensor signal to the control unit 44. The sensor unit 44 is configured to actuate a drive unit, not visible in FIG. 2, for moving the closure element 60 as a function of the transmitted sensor signal.

The bill dispensing unit 16 furthermore comprises a transport unit 70 with the aid of which bills 48 retrieved from the strongbox module 14 are transported in a direction T1 to the dispensing compartment 72. Those bills not retrieved from the dispensing compartment, with the aid of the transport unit 70, are transported in a direction counter to the direction T1.

The closure element 60 is a first inner closure element of the device 10 which as a “shadow” closure element 60 is disposed behind a second outer closure element, not illustrated. The outer closure element can be opened and closed independently of the closure element 60. In this way, the retrieval of bills 48 from the bill dispensing unit 16 is possible only when both closure elements 60 are simultaneously opened. A particularly high degree of security is achieved as a result because the “shadow” closure element 60 remains closed in particular when the outer closure element is manipulated by any unauthorized third party.

FIG. 3 shows a sectional illustration of a fragment of the bill dispensing unit 16 as per FIG. 2 along the section plane A. During a disbursement procedure, the bills 48 are first transported in the direction of the dispensing compartment 72 by way of two transport belts which are in each case deflected by two deflection rollers, wherein the transport belt 73 and the deflection roller 71 are visible in the sectional illustration as per FIG. 3. Subsequently, the bills 48 are fed to further transport elements 80 to 90 with the aid of which the bills 48 are fed, preferably individually, to the dispensing compartment 82. The transport elements 80 to 90 comprise transport rollers, rotary blades and contact pressure rollers, wherein the transport roller 84 disposed on the shaft 80, the transport roller 86 disposed on the shaft 82, and the rotary blade 88 as well as the contact pressure roller 90 are visible in the illustration as per FIG. 3. The shafts 80 and 82 are interconnected by a gearbox assembly not visible in FIG. 3 and are driven with the aid of the drive unit 76.

When feeding the bills 48 into the dispensing compartment 72, the drive unit 76 is actuated by the control unit 44 in such a manner that the shafts 80 and 82 are in each case driven in a first rotating direction. In particular, the shaft 80 rotates in a counter-clockwise manner, and the shaft 82 correspondingly rotates in a clockwise manner, so that the bill 48 is fed to the dispensing compartment 72.

If the bills 48 are not retrieved from the device 10, the so-called retracts from the dispensing compartment 72 are fed to the reject and retract container 54. The retrieval takes place with the aid of discharge rollers 92 which ensure that the bills 48 are retrieved, preferably individually or alternatively as a stack, from the dispensing compartment 72. When retrieving the bills from the dispensing compartment 72, the shafts 80 and 82 are in each case driven in a second rotating direction. In particular, the shaft 80 rotates in a clockwise manner, and the shaft 82 correspondingly rotates in a counter-clockwise manner, so as to retrieve the bill 48 from the dispensing compartment 72.

The dispensing compartment 72 on a first side is delimited by the depositing element 62, and on an opposite second side is delimited by a delimiting element 74. The delimiting element 74 comprises two contact pressure elements 96, 97 for the orderly depositing of the bills 48, wherein only the contact pressure element 96 is visible in the illustration as per FIG. 3 while the second contact pressure element 97 is illustrated in FIGS. 4, 6, 9 and 10. The description hereunder pertaining to the contact pressure element 96 applies in an analogous manner also to the contact pressure element 97 which in terms of construction and function is identical to the contact pressure element 96.

The contact pressure element 96 is preferably disposed so as to be pivotable on the delimiting element 76 and is connectable to the latter. No bills 48 are deposited in the dispensing compartment 72 in the illustration as per FIG. 3. The delimiting element 74 is situated in a first delimiting position, and the contact pressure element 96 is situated in a first contact pressure position, wherein the contact pressure element 96 contacts the depositing element 62. When a bill 48 is fed to the dispensing compartment 72, the movement of the bill 48 is decelerated or stopped by the contact pressure element 96. Moreover, the fed bill 48 deflects the contact pressure element 96 in the direction of the delimiting element 74. The fed bill 48, already when being fed into the dispensing compartment 72, by the contact pressure element 96 is pushed against the depositing element 62, thus ensuring an orderly depositing of the bills.

The fed bills 48 form a bill stack, wherein the bills 48 of the bill stack are stacked on top of one another in such a manner that the lowermost, i.e. the first fed, bill 48 by way of the front side thereof or the rear side thereof bears on the depositing element 62. The uppermost bill 48 by way of the front side thereof or the rear side thereof contacts the contact pressure element 96. The direction from the lowermost bill 48 to the uppermost bill 48 is a stacking direction R1 of the bill stack. The more bills 48 deposited on the depositing element 62, the higher the bill stack formed by the bills 48 and the greater the deflection of the contact pressure element 96 in the direction of the delimiting element 74. Visible in the illustration as per FIG. 3 is a sensor unit 98 which is configured to detect a second contact pressure position of the contact pressure element 96 that corresponds to a pre-set deflection of the contact pressure element 96 and thus to a pre-set height of the bill stack.

The sensor unit 98 preferably comprises a light barrier having a transmitter and having a receiver which are disposed opposite one another. The sensor unit 98, which is assigned to the contact pressure element 96, and a further sensor unit 99, which is assigned to the contact pressure element 97, will be explained in more detail in the context of FIG. 6. When the contact pressure element 96 is deflected to the second contact pressure position, an interrupter element not visible in FIG. 3 interrupts the light beam emitted by the transmitter of the sensor unit 98, the receiver detecting the interruption. As soon as the interruption of the light beam of the sensor unit 98, or of the light beams of both sensor units 98, 99, is detected by the sensor unit 98, the latter generates a detection signal and transmits the detection signal to the control unit 44. The control unit 44 thereupon actuates a drive unit 94 which by way of a gearbox assembly 95 moves the depositing element 62 by a pre-set distance counter to the stacking direction R1.

As a result of this movement, the contact pressure element 96 is moved from the second contact pressure position, away from the delimiting element 74, so that the second contact pressure position of the contact pressure element 96 is no longer detected by the sensor unit 98. Subsequently, further bills 48 can be fed to the dispensing compartment 72, wherein the procedure of the movement of the depositing element 62 counter to the direction R1 described above is repeated when the contact pressure element 96 is moved yet again to the second contact pressure position by the deposited bills 48.

The depositing element 62 comprises a plurality of ribbed elements of which one ribbed element identified by the reference sign 100 is visible in the illustration as per FIG. 3. In the closed position of the closure element 60 illustrated in FIG. 3 the ribbed element 100 engages in a complementary clearance of the closure element 60. As a result, access to the dispensing compartment 72 by any unauthorized third party is impeded. The delimiting element 74 furthermore comprises an engagement element 102 which is movable between a deployed position, illustrated in FIG. 3, and a retracted position.

As soon as all bills 48 destined for disbursement have been fed to the dispensing compartment 72, the control unit 44 performs steps for opening the dispensing compartment 72, said steps yet to be described in detail hereunder in the context of FIG. 12. The dispensing element 62 herein, with the aid of the drive unit 94, is moved in the direction of the delimiting element 74, wherein the delimiting element 74 is disposed in such a manner that the latter in this movement of the depositing element 62 is moved to a second delimiting position. Furthermore, the control unit actuates a further drive unit 104, not visible in the illustration as per FIG. 3, so as to move the closure element 60 from the closed position, illustrated in FIG. 3, to an opened position. During the movement from the closed to the opened position, the closure element 60 comes to engage with the engagement element 102 of the delimiting element 74 disposed in the second delimiting position.

Upon engaging with the closure element 60, the engagement element 102 and the delimiting 74 are moved conjointly with the closure element 60 until the closure element 60 assumes the opened position illustrated in FIGS. 4 and 5. In the course of this conjoint movement of the delimiting element 74 and the closure element 60, the delimiting element 74 is moved from the second delimiting position to a third, opened delimiting position.

During the movement from the closed position to the opened position, the closure element 60 is guided in a guide track, a movement of the closure element 60 about a virtual rotation axis D1 being in particular performed herein. In contrast, the delimiting element 74 is rotated about a second rotation axis D2, which is different from the rotation axis D1 and is congruent with the longitudinal axis of the shaft 80. In this way, the pivot radius S1 of the closure element 60 is larger than the pivot radius S2 of the delimiting element 74. As a result of the described disposal of the closure element 60 and of the delimiting element 74, it is achieved that the engagement element 102 is moved from the deployed to the retracted position, while the delimiting element 74 is moved from the second delimiting position to the third delimiting position. The engagement element 102 herein deforms an elastic element, preferably a restoring spring, which is not visible in the illustration as per FIG. 3.

Furthermore, the control unit 44, during the movement of the delimiting element 74 from the second delimiting position to the third delimiting position, is configured to actuate the drive unit 94 so as to move the depositing element 62 in the direction counter to the delimiting element 74. It is achieved as a result that the volume of the dispensing compartment 72 in the case of an opened closure element 60 is further enlarged so that ready access to the bill 48 or bill stack situated in the dispensing compartment 72 is possible. The position of the dispensing element is monitored with the aid of a sensor unit 140, wherein the control unit is configured to actuate the drive unit 94 as a function of a signal of the sensor unit 140.

FIG. 4 shows a perspective detailed view of the bill dispensing unit 16 as per FIGS. 2 and 3, having an opened closure element 60. In addition to the transport elements illustrated in FIG. 3, the transport rollers 84a and 86a, the rotary blades 88a and 88b, as well as the discharge roller 92a and the second depositing element 97 are also visible in the illustration as per FIG. 4. Moreover visible is part of the gearbox assembly 106. Furthermore visible is a second clearance 164 which is disposed so as to be opposite the clearance 64 and further facilitates access to the bills 48 in that an operator can reach below the bills 48 in the clearance 64 with his/her fingers and hold the bills 48 from above in the clearance 164 with his/her fingers. In the opened position of the closure element 60 shown in FIG. 4, access to the bills 48 deposited in the dispensing compartment 72 is readily possible when the outer closure element, not illustrated, is also simultaneously opened.

FIG. 5 shows a fragment of a perspective sectional illustration of the bill dispensing unit 16 along the section plane B as per FIG. 4. The engagement between the engagement element 102 and the closure element 60 is particularly visible in the illustration as per FIG. 5, the delimiting element 74 being held in the third delimiting position by said engagement.

FIG. 6 shows a perspective view of the delimiting element 76 as per FIGS. 2 to 5. For illustrative purposes, the contact pressure element 96 is illustrated in the first contact pressure position and the contact pressure element 97 is illustrated in the second contact pressure position. However, during a disbursement procedure, the contact pressure elements 96, 97 always assume identical contact pressure positions. An interrupter element 115 of the contact pressure element 97 interrupts the light beam emitted by the transmitter 110 of the sensor unit 99, said light beam thus no longer being detected by the receiver 111 so that the second contact pressure position of the contact pressure element 97 is detected by the control unit 44. However, in the first contact pressure position of the contact pressure element 96, the light beam between the transmitter 112 and the receiver 113 is not interrupted by the interrupter element 114. Furthermore visible in the illustration as per FIG. 6 are the springs 106 and 108 which are elastically deformed by the respective engagement elements 102 and 103 when the latter are moved from the deployed to the retracted position.

FIG. 7 shows a perspective sectional illustration of several elements of the bill dispensing unit 16 as per FIGS. 2 to 6, in which the delimiting element 74 is situated in the second delimiting position and the closure element 60 is situated between the closed position and the opened position.

FIG. 8 shows an enlarged detailed view of part of the delimiting element 76 of the engagement element 102 and part of the closure element 60 as per FIG. 7. In the illustration as per FIG. 8 the delimiting element 74 is disposed in the second delimiting position. A tip 130 of the engagement element 102 that projects from the delimiting element 74 in the direction of the closure element 60 protrudes into the motion path of a protrusion 132 of the closure element 60. In a further movement of the closure element 60 in the direction R2, the protrusion 132 and the tip 130 come to mutually engage in such a manner that the engagement element 102 is entrained by the closure element 60 so that the delimiting element 74, conjointly with the engagement element 102, is pivoted about the rotation axis D2 of said engagement element 102.

FIG. 9 shows a perspective detailed view of the depositing element 62 and of the delimiting element 74. FIG. 10 shows a further perspective detailed view of the bill dispensing unit 16. Discharge means 93, 93a, 93b, 93c which are disposed on the discharge rollers 92, 92a and which are preferably made from an elastomer and ensure the singularizing of the bills 48 that are not retrieved from the dispensing compartment 72 and classified as retracts are particularly visible in the illustration as per FIG. 10.

FIG. 11 shows a further perspective detailed view of the bill dispensing unit 16 as per FIGS. 2 to 10, having the transport unit 70 for feeding the bills 48 into the dispensing compartment 72 and for transporting the bills 48 not retrieved in the direction of the reject and retract container 54. During the disbursement procedure, the bills 48 from the strongbox module 14 are fed to the transport unit 70 by way of the transfer opening 18 and transported in the direction T1 to the dispensing compartment 72. Bills 48 that are not retrieved are transported from the dispensing compartment 72 in the direction T2, counter to the direction T1. The transport path 120 of the bills 48 on a first side is delimited by the transport belt 73 and a base element 122, and on the opposite side is delimited by a cover element 124.

FIG. 12 shows a further schematic illustration of the bill dispensing unit 16 as per FIGS. 2 to 11, in which sensor units 66, 68, 98, 99, 140, 142 for controlling the infeed of bills into the dispensing compartment 72 and for dispensing bills are schematically illustrated in particular.

For feeding the bills 48 into the dispensing compartment 72, the dispensing element 62 with the aid of the drive unit 94 is displaced to a first feeding position. For displacing the depositing element 62 to the first feeding position, the control unit 44 in a first step is configured to actuate the drive unit 94 for moving the depositing element 62 as a function of the sensor signals emitted by the sensor units 98 and 99. The depositing element 62 herein, from an initial position, for example a lower position that can be detected with the aid of the sensor unit 140, is displaced upward in the direction of the delimiting element 74 until the depositing element 62 contacts the contact pressure elements 96, 97 which are articulated on the delimiting element 74 and by the depositing element 62 are pushed to the second contact pressure position. The second contact pressure position is detected with the aid of the sensor units 98 and 99, the latter transmitting the sensor signal to the control unit 44.

At the point in time at which the second contact pressure position of the contact pressure elements 96, 97 is detected by the sensor units 98, 99, the depositing element 62 is disposed in an initializing position. Proceeding from the reception of the sensor signals of the sensor units 98, 97, the control unit actuates the drive unit 94 in such a manner that the depositing element 62 is moved by a pre-set distance away from the delimiting element 74, from the initializing position to a first feeding position for feeding the bills 48. The distance is predefined in particular by a pre-set number of steps of a stepper motor of the drive unit 94. Another drive unit may also be provided in other embodiments, said other drive unit comprising in particular measuring the distance of travel of the depositing element 62.

As soon as the depositing element 62 is situated in the feeding position, bills 48 can be transported into the dispensing compartment 72 and deposited therein. The control unit 44, preferably from the point in time from which the sensor 42 detects a bill 48 cleared for disbursement and transmits the corresponding sensor signal to the control unit 44, actuates the drive unit 76 in such a manner that feeding of the bills 48 into the dispensing compartment 72 is possible.

A further sensor unit 142, preferably an analog light barrier assembly, which determines whether at least one bill 48 to be disbursed is deposited in the dispensing compartment 72, or whether the dispensing compartment 72 is empty, is provided for monitoring the dispensing compartment 72. A corresponding sensor signal is transmitted to the control unit 44 when a bill 48 is detected in the dispensing compartment 72.

Once the bill stack of the bills 48 deposited in the dispensing compartment 72 reaches a predetermined height and the depositing element 62 is situated in the first feeding position, the contact pressure elements 96, 97 are deflected or pivoted, respectively, to the second contact pressure position, the sensor units 98, 99 herein transmitting the corresponding sensor signal to the control unit 44.

Proceeding from the detection of the sensor signal of the sensor units 98 and 99 and the detection of the sensor signal of the sensor 142, the control unit 44 actuates the drive unit 94 for moving the depositing element 62 in such a manner that the depositing element 62 is moved away from the delimiting element 74, from the first feeding position to a second feeding position. This procedure can preferably be repeated twice, that is to say that a third feeding position of the depositing element 62 may also be provided. More than three or fewer than three feeding positions may also be provided in alternative embodiments.

Once all bills 48 provided for disbursement have been transported into the dispensing compartment 72, the dispensing compartment 72 is opened so that an operator can access the bills 48 provided for retrieval in the dispensing compartment 72. For opening the dispensing compartment 72, the control unit 44 in a first step is configured to actuate the drive unit 94 in such a manner that the depositing element 62, proceeding from the initial position in which the depositing element 62 is situated, is moved to an entrainment position. The initial position may be the first feeding position, the second feeding position, the third feeding position, or else the lower position of the depositing element 62 that can be detected with the aid of the sensor unit 140.

The entrainment position is shown in an exemplary manner in FIGS. 7 and 8 and identifies a position of the depositing element 62 in which the delimiting element 74 by the depositing element 62, or by the bills 48 deposited on the depositing element 62, respectively, is raised to the second delimiting position, specifically in such a manner that, when opening the closure element 60, the engagement elements 102, 103 of the delimiting element 74 each come to engage with a protrusion 132 of the closure element 60 so that the delimiting element 74, conjointly with the engagement element 102, is pivotable about the rotation axis D2 of said engagement element 102.

In a second step, the control unit 44 actuates the drive unit 144 for opening the closure element 60, wherein the engagement elements 102, 103 and the delimiting element 74 in the course of the opening movement of the closure element 60 come to engage with the closure element 60 and are moved conjointly with the closure element 60 until the closure element 60 assumes the opened position illustrated in FIGS. 4 and 5. In the course of this conjoint movement of the delimiting element 74 and the closure element 60, the delimiting element 74 is moved from the second delimiting position to the third, opened delimiting position and is held in the opened position by the closure element 60.

The control unit 44 preferably actuates the drive unit 144 for opening the closure element 60 as a function of the sensor signal of the sensor units 66 and 68. The sensor unit 66 determines the closed position, and the sensor unit 68 determines the opened position of the closure element 60. The control unit 44 actuates the drive unit 144 in particular in such a manner that the closure element 60 for retrieving the bills 48 remains in the opened position for a predetermined period, and after the pre-set period is moved to the closed position.

Bills 48 that have not been retrieved from the dispensing compartment 72 have to be transported as so-called retracts into the reject and retract container 54. For this purpose, the control unit 44, after the pre-set period has elapsed, is configured to check whether the sensor unit 142 relays the sensor signal to the control unit 44. Should this condition be met, the control unit 44 actuates the drive unit 94 in such a manner that the depositing element 62 is moved from the entrainment position to a discharge position in which the bills 48 are retrievable from the dispensing compartment with the aid of the discharge rollers 92, 92a. Furthermore, the control unit 44 actuates the drive unit 76 in such a manner that the discharge rollers 92, 92a are driven so as to transport the bills 48 into the reject-retract container 54.

FIG. 13 shows a perspective view of a bill dispensing unit 116 according to a second embodiment which, as an alternative to the bill dispensing unit 16 as per FIGS. 2 to 12, is suitable for use in the device 10 as per FIG. 1. Elements of identical construction or identical function have the same reference signs.

The illustration as per FIG. 13 is a perspective detailed view of the bill dispensing unit 116 having an opened closure element 60. The bill dispensing unit 116 in terms of construction and function is identical to the bill dispensing unit 16, but the contact pressure elements 96, 97 have been replaced by the contact pressure elements 196 and 197. Other aspects of the construction and other functions are identical.

As opposed to the contact pressure elements 96, 97, the contact pressure elements 196, 197 are not connected to the delimiting element 76 but are disposed so as to be pivotable about a fixed rotation axis (D3 in FIG. 18) so that the contact pressure elements 196, 197 are movable independently of the delimiting element 96. In FIG. 13, a bill 28 is disposed in the dispensing compartment 72, said bill 28 being held on the depositing element 62, or being pushed against the depositing element 62, respectively, by the contact pressure elements 196, 197. In the position of the depositing element 62 shown in FIG. 13, the contact pressure elements 196, 197 are pushed against the depositing element 62 with the aid of springs 220, 221. The contact pressure element 196 for guiding the movement has a guide bar 216 which protrudes into a guide opening 218 and in a movement of the contact pressure element 196 is guided counter to the spring force and in the direction of the spring force. The spring 220 is configured as a spiral spring and is disposed about the guide bar 216. One end of the spring 220 pushes against the contact pressure element 196. The other end of the spring pushes against a fixed housing part of the dispensing compartment 62. In the same way, the spring 221 on the contact pressure element 197 is disposed about a guide bar 217, wherein the guide bar 217 protrudes into a guide opening 219 and in a movement of the contact pressure element 197 is guided in the guide opening 219.

FIG. 14 shows a perspective detailed view of a fragment of the bill dispensing unit as per FIG. 13. A plurality of elements have been omitted for the purpose of simplified illustration and improved clarity. As opposed to the illustration as per FIG. 13, the delimiting element 76 is disposed in a lower position. Furthermore, no bill is disposed in the dispensing compartment 72. Despite the change of the position of the delimiting element 76, the contact pressure elements 196, 197 are unchanged in comparison to FIG. 13. The contact pressure elements 196, 197 are raised when a plurality of bills are stacked on the depositing element 62. The contact pressure elements 196, 197 each have an interrupter element 214, 215. Once a predetermined stack height of the bills 28 stacked on the depositing element has been reached, the interrupter elements 214, 215 drop into the detection range of a sensor unit 198, 199 configured as a light barrier assembly, interrupting the light beam between the transmitter and the receiver (both not illustrated). As a result, the control unit 44 by way of a sensor signal of the sensor units 198, 199 is informed that a predetermined stack height of the bills situated in the dispensing compartment 72 has been reached. This signal can also be utilized for setting the height of the depositing element 62 when initializing the bill dispensing unit 116, as has been described for the bill dispensing unit 16 in the context of the contact pressure elements 96, 97 and the sensor units 98, 99.

In the same way as described for the contact pressure elements 96 and 97 of the bill dispensing unit 16, the contact pressure elements 196, 197 are situated in a first contact pressure position, and the delimiting element 74 is situated in a first delimiting position, before bills are deposited in the dispensing compartment 72 by the bill dispensing unit 116. The contact pressure element 196, 197 herein contacts the depositing element 62. When a bill 28, 48 is fed to the dispensing compartment 72, the movement of the bill 48 is decelerated or stopped by the contact pressure elements 196, 197. Moreover, the fed bill 48 deflects the contact pressure element 96 in the direction of the sensor unit 198, 199. The fed bill 48, already when being fed into the dispensing compartment 72, by the contact pressure elements 196, 197 is pushed against the depositing element 62, thus ensuring an orderly depositing of the bills 28, 48.

The fed bills 28, 48 form a bill stack, wherein the uppermost bill of the bill stack by way of the front side thereof or the rear side thereof contacts the contact pressure elements 196, 197. The more bills deposited in the dispensing compartment 72, the greater the deflection of the contact pressure element 196, 197 in the direction of the sensor unit 198, 199. A second contact pressure position of the contact pressure elements 196, 197 is detected with the aid of the sensor units 198, 199, said second contact pressure position corresponding to a pre-set deflection of the contact pressure elements 196, 197 and thus to a pre-set height of the bill stack. To this end, the respective interrupter element 214, 215 interrupts the light beams emitted by the transmitter of the sensor units 198, 199, wherein the respective receiver detects the interruption, as a result of which a detection signal is transmitted to the control unit 44.

Proceeding therefrom, the control unit 44 actuates the drive unit 94 which by way of a gearbox assembly 95 moves the depositing element 62 by a pre-set distance counter to the stacking direction R1. As a result of this movement, the contact pressure elements 196, 197 are moved from the second contact pressure position, away from the sensor units 198, 199, so that the second contact pressure position of the contact pressure elements 196, 197 is no longer detected by the sensor units 198, 199. Subsequently, further bills can be fed to the dispensing compartment 72, wherein the procedure of the movement of the depositing element 62 counter to the direction R1 described above is repeated when the contact pressure elements 196, 197 are moved yet again to the second contact pressure position by the deposited bills.

FIG. 15 shows the fragment of the bill dispensing unit 116 as per FIG. 14, wherein the delimiting element 74 of the bill dispensing unit 116 has been omitted.

FIG. 16 shows a perspective detailed view of the depositing element 74 and of contact pressure elements 196, 197 of the bill dispensing unit 116 as per FIGS. 13 to 15. In the present exemplary embodiment, the contact pressure elements 196, 197 are disposed so as to be pivotable about a pivot axis D3 illustrated in FIG. 17, and the contact pressure element 74 is disposed so as to be pivotable about a pivot axis D2 shown in FIG. 6. In the present exemplary embodiment, the pivot axes D2 and D3 are mutually parallel and spaced apart. In other exemplary embodiments, the rotation axes D2 and D3 may also lie on a straight line so that the contact pressure elements 196, 197 and the delimiting element 74 are pivoted about the same rotation axis. Provided in the bill dispensing unit 116 are further spring elements 222, 223 which exert on the delimiting element 74 a force in the direction of the depositing element 62 so that the delimiting element 74 is securely held in a lower position when bills are deposited in the dispensing compartment 72 or the closure element 60 is closed. As can be seen in FIG. 16, the interrupter elements 214, 215 are guided by openings 224, 226 in the delimiting element 74. The interrupter elements 214, 215 in terms of the movement thereof relative to the delimiting element 74 are in each case guided by guide elements 227, 228 of the contact pressure elements 196, 197, wherein a protrusion that serves as a detent is configured on that end of the guide element 227, 228 that is opposite the contact region with the bill 28, 48 of the respective contact pressure element 196, 197. The pivoting movement of the contact pressure elements 196, 197 in relation to the delimiting element 74 is delimited by the protrusion when the protrusion contacts the delimiting element 74.

FIG. 17 shows a further perspective detailed view of the dispensing element 74 as per FIG. 16, without contact pressure elements 196, 197.

FIG. 18 shows a perspective detailed view of a fragment of the contact pressure elements 196, 197 as per FIG. 16.

FIG. 19 shows a flow chart of a method for initializing the bill dispensing unit 16, 116 as per one of FIGS. 2 to 18. Proceeding from the flow chart as per FIG. 19, FIG. 20 shows a signal diagram for controlling the initialization of a bill dispensing unit 16, 116 as per one of FIGS. 2 to 18.

The initialization sequence is started by step S1. Subsequently, the lower position of the depositing element 62 is detected with the aid of the sensor unit 140. To this end, the depositing element 62 with the aid of the drive unit 96 for moving the depositing element 62 is moved down to the lower position, if required. Subsequently, in step S3, the depositing element 62 with the aid of the drive unit 94 is displaced in the direction of the delimiting element 74. In step S4, it is then checked whether the depositing element 62 is displaced so far in the direction of the delimiting element 74 to the point that the depositing element 62 has deflected the contact pressure elements 96, 97, 196, 197 so far that the sensor units 98, 99, 198, 199 detect the respective interrupter element 214, 125, 114, 115. When both sensor units 98, 99, or 198, 199, respectively, detect a second upper position of the contact pressure elements 96, 97, 196, 197, the movement of the depositing element 62 in the direction of the delimiting element 74 is interrupted. In step S5, the depositing element 62 with the aid of the drive unit 94 is subsequently moved by a predetermined distance away from the delimiting element 74 again so that the depositing element 62 is then disposed in the feeding position in which depositing bills in the depositing compartment 72 is possible. The initialization sequence is then completed in step S6.

In the signal diagram shown in FIG. 20, the actuation of the drive unit 94 for moving the depositing element 62 is shown in the top line. The signal profile of the sensor unit 140 relevant to step S2 is illustrated in line 2; the signal profile of the sensor units 98, 198 relevant to steps S3 and S4 is shown in line 3; the signal profile of the sensor units 99, 199 relevant to steps S3 and S4 is shown in line 4; and the signal profile of the signal upon reaching the feeding position generated by the control unit 44 is shown in line 5.

At the time T1, the drive unit 94 for moving the depositing element 62 is actuated in such a manner that the depositing element 62 is displaced downward to the lower position. Once it has been detected with the aid of the sensor unit 140 at the time T2 (step S2) that the lower position of the depositing element 62 has been reached, the depositing element 62 with the aid of the drive unit is displaced in the direction of the delimiting element 74 (step S3) until the sensor unit 98, 99, or 198, 199, respectively, each detect a second position of the depositing elements 96, 97, or 196, 197, respectively, (step S4) and the movements of the depositing element 62 are stopped at the time T3. Subsequently, the depositing element 62 is moved in the direction of the lower position, away from the delimiting element 74 again. Once the sensor units 98, 99, or 198, 199, respectively, at the time T4 no longer detect the second position of the contact pressure elements 96, 97, 196, 197 at the time T4, the depositing element 62 is moved further downward by a pre-set distance. In the present exemplary embodiment, the latter corresponds to 21 steps of the stepper motor serving as the drive of the depositing element 62. Upon performing the controlled movement at the time T5, the control unit 46 generates a signal which indicates that the depositing element 62 has reached the feeding position for feeding bills into the depositing compartment 72.

### LIST OF REFERENCE SIGNS

10 Device
12 Head module
14 Strongbox module
16, 116 Bill dispensing unit
18 Transfer opening
20 to 26 Bill cassette
28, 48, 50 Bill
30 to 36 Singularizing unit
40 Transport path
42, 66, 68, 98, 99, 140
142, 144, 198, 199 Sensor unit
44 Control unit
46 Turnout unit
52 Intermediate storage
54 Reject and retract container
60 Closure element
62 Depositing element
64, 164 Clearance
70 Transport unit
71 Deflection roller
72 Dispensing compartment
73 Transport belt
74 Delimiting element
76, 94, 144 Drive unit
80, 82 Shaft
92, 92a Discharge roller
84, 84a, 86, 86a Transport roller
88, 88a, 88b Rotary blade
90 Contact pressure roller
93, 93a, 93b, 93c Discharge means
95 Gearbox assembly
96, 97, 196, 197 Contact pressure element
100 Ribbed element
102, 103 Engagement element
106, 108, 220, 221,
222, 223 Spring
114, 115, 214, 215 Interrupter element
110, 112 Transmitter
111, 113 Receiver
122 Base element
124 Cover element
130 Tip
132 Protrusion
216, 217 Guide bar
218, 219 Guide opening
224, 226 Opening
227, 228 Guide element
D1, D2, D3 Rotation axis
R1, R2, T1 Direction
S1, S2 Pivot radius

1. A device for handling bills comprising:
a dispensing compartment which includes a depositing element that is moveable and delimits the dispensing compartment on a first side and a delimiting element that delimits the dispensing compartment on a second side;
a control unit;
at least one first sensor unit configured to detect when the depositing element is in an initializing position and configured to generate a first sensor signal and to transmit the first sensor signal to the control unit;
a first drive unit configured to move the depositing element;
wherein the control unit is configured to actuate the first drive unit to move the depositing element; and
wherein the control unit, in response to the first sensor signal, actuates the first drive unit such that the depositing element is moved a predetermined distance away from the delimiting element, from the initializing position to a first feeding position for feeding the bills.

2. The device as claimed in claim 1 wherein the at least one first sensor unit further comprises at least one sensor element that is moveable, wherein the at least one first sensor unit is disposed in such a manner that a first position of the at least one sensor element is detectable by the at least one first sensor unit, and wherein the at least one first sensor unit is configured to emit the first sensor signal as a function of detecting at least one sensor element at the first position.

3. The device as claimed in claim 2 wherein the at least one first sensor unit detects the first position of the at least one sensor element when the depositing element is in the initializing position, wherein the at least one sensor element contacts the depositing element when the depositing element is in the initializing position and moves in the direction of the delimiting element as a result of movement of the depositing element in the direction of the delimiting element.

4. The device of claim 2 wherein the at least one sensor element is a contact pressure element pivotably connected to the delimiting element and wherein the at least one sensor element is movable between the first position and a second position, wherein the first position is lower than the second position.

5. The device of claim 1 further comprising:
at least one second sensor unit configured to detect the depositing element when the depositing element is in a lower position and configured to generate a second sensor signal in response to detecting the depositing element when the depositing element is in the lower position and also configured to transmit the second sensor signal to the control unit, wherein the control unit is configured to actuate the first drive unit to move the depositing element in response to the second sensor signal.

6. The device as claimed in claim 5 wherein the control unit, in response to the second sensor signal, is configured to actuate the first drive unit to move the depositing element a predetermined distance toward the delimiting element, to the initializing position.

7. The device of claim 1 further comprising:
a closure element moveable between a closed position whereby the closure element delimits and closes the dispensing compartment on a third side and an opened position whereby bills in the dispensing compartment are accessible by an operator.

8. The device as claimed in claim 7 further comprising:
at least one third sensor unit configured to detect the closure element when the closure element is in the closed position;
at least one fourth sensor unit configured to detect the closure element when the closure element is in the opened position;
a second drive unit;
wherein the at least one third sensor unit is further configured to generate a third sensor signal and to transmit the third sensor signal to the control unit;
wherein the fourth sensor unit is further configured to generate a fourth sensor signal and to transmit the fourth sensor signal to the control unit; and
wherein the control unit is further configured to actuate the second drive unit to drive the closure element in response to one at least one of the third sensor signal and the fourth sensor signal.

9. The device of claim 1 further comprising:
a fifth sensor unit disposed in the dispensing compartment and configured to generate a fifth sensor signal and to transmit the fifth sensor signal to the control unit when at least one bill is received in the dispensing compartment.

10. The device of claim 2 wherein:
the dispensing compartment is configured to receive a plurality of bills thereby forming a bill stack, wherein the plurality of bills of the bill stack are disposed on top of one another in such a manner that a lowermost bill of the plurality of bills by way one of the front side thereof or the rear side thereof bears on the depositing element and that an uppermost bill of the plurality of bills by way of one the front side thereof or the rear side thereof contacts the at least one sensor element;
the direction from the lowermost bill to the uppermost bill is a stacking direction of the bill stack;
the at least one first sensor unit detects the at least one sensor element position when the bill stack contacts the at least one sensor element; and
the sensor element moves in the direction of the delimiting element as a result of contacting the bill stack.

11. The device of claim 10 further comprising:
a fifth sensor unit disposed in the dispensing compartment and configured to generate a fifth sensor signal and to transmit the fifth sensor signal to the control unit when at least one bill is received in the dispensing compartment;
wherein the control unit, in response to receiving the first sensor signal and the fifth sensor signal, actuates the first drive unit to move the depositing element a predetermined distance away from the delimiting element, from the first feeding position to a second feeding position, such that the dispensing compartment is operable to receive further bills.

12. The device of claim 1 further comprising:
a closure element moveable between a closed position whereby the closure element delimits and closes the dispensing compartment on a third side and an opened position whereby bills in the dispensing compartment are accessible by an operator;
at least one third sensor unit configured to detect the closure element when the closure element is in the closed position and configured to generate a third sensor signal;
a second drive unit;
at least one engagement element mounted on the delimiting element;
wherein the control unit is configured to actuate the first drive unit to move the depositing element from one of a lower position of the depositing element, the first feeding position, and a second feeding position of the depositing element, a predetermined distance to an entrainment position;
wherein the delimiting element is moved from a first delimiting position to a second delimiting position by the movement of the depositing element to the entrainment position;
wherein the control unit is further configured, in response to receiving the third sensor signal, to actuate the second drive unit to move the closure element from the closed position to the opened position; and
wherein the at least one engagement element engages an engagement region of the closure element.

13. The device of claim 1 further comprising:
at least one second sensor unit configured to detect the depositing element when the depositing element is in a lower position and configured to generate a second sensor signal and to transmit the second sensor signal to the control unit;
a closure element moveable between a closed position whereby the closure element delimits and closes the dispensing compartment on a third side and an opened position whereby bills in the dispensing compartment are accessible by an operator;
at least one third sensor unit configured to detect the closure element when the closure element is in a closed position and configured to generate a third sensor signal and to transmit the third sensor signal to the control unit;
at least one fourth sensor unit configured to detect the closure element when the closure element is in the opened position and configured to generate a fourth sensor signal and to transmit the fourth sensor signal to the control unit;
a fifth sensor unit disposed in the dispensing compartment and configured to generate a fifth sensor signal and to transmit the fifth sensor signal to the control unit when at least one bill is received in the dispensing compartment; and
wherein at least one of the at least one first sensor unit, the at least one second sensor unit, the at least one third sensor unit, the at least one fourth sensor unit, and the at least one fifth sensor unit further comprises at least one detection element, wherein the at least one detection element generates the respective sensor signal and transmits the respective sensor signal to the control unit, and wherein the at least one detection element comprises a light barrier.

14. The device of claim 1 further comprising:
a plurality of transport elements configured to transport bills into and out of the dispensing compartment;
at least one third drive unit configured to drive the plurality of transport elements, wherein the plurality of transport elements are drivable in a first direction to move the bills to the dispensing compartment, and wherein the plurality of transport elements are drivable in a second direction, counter to the first direction, to move the bills out of the dispensing compartment;
a sixth sensor unit configured to detect at least one feature of the bills, and configured to generate a sixth sensor signal and to transmit the sixth sensor signal to the control unit, wherein the control unit is configured to distinguish whether at least one bill of the bills is cleared for disbursement in response to the sixth sensor signal unit; and
wherein the control unit controls the third drive unit to drive the plurality of transport elements in the first direction when the sixth sensor unit detects the at least one bill cleared for disbursement.

15. The device of claim 1 further comprising:
a closure element moveable between a closed position whereby the closure element delimits and closes the dispensing compartment on a third side and an opened position whereby bills in the dispensing compartment are accessible by an operator;
a second drive unit configured to move the closure element from the closed position to the opened position;
a fifth sensor unit disposed in the dispensing compartment and configured to generate a fifth sensor signal and to transmit the fifth sensor signal to the control unit when at least one bill is received in the dispensing compartment;
wherein the control unit is configured to actuate the second drive unit such that the closure element is disposed in the opened position for a predetermined period and after the predetermined period is moved to the closed position;
wherein the control unit, after the predetermined period has elapsed, is configured to check whether the fifth sensor unit has transmitted the fifth sensor signal to the control unit;
wherein the control unit, should the fifth sensor signal has been relayed to the control unit, is configured to actuate the first drive unit such that the depositing element is moved from an entrainment position to a discharge position; and
wherein the control unit is configured to actuate the third drive unit such that the transport elements are driven in the second direction.

16. The device of claim 1 further comprising:
a closure element pivotally moveable about a first rotation axis between at least a closed position and an opened position;
wherein said depositing element is further defined as moveable along a rectilinear path of movement between at least a lower position and the initializing position;
wherein said delimiting element is further defined as pivotally moveable about a second rotation axis through at least a first delimiting position, a second delimiting position, and a third delimiting position; and
wherein said first rotation axis and said second rotation axis are spaced from one another.

17. The device of claim 16 wherein:
said closure element further comprises at least one protrusion a first distance from said first rotation axis and thereby moves along a first arcuate path as said closure element moves between said closed position and said opened position;
said delimiting element further comprises at least one engagement element with a distal tip that extends a second distance from said second rotation axis when said delimiting element is at said first delimiting position and said second delimiting position; and
said first arcuate path and said second distance intersect when said delimiting element is at said second delimiting position, whereby said distal tip is received in said at least one protrusion.

18. The device of claim 17 wherein at least part of said at least one engagement element is elastically deformable whereby said distal tip is retractable into a remainder of said at least one engagement element and said second distance is a maximum distance between said distal tip and said second rotation axis.

19. The device of claim 18 wherein said at least one engagement element further comprises:
a spring biasing said distal tip outward from said remainder of said at least one engagement element, to extend to said second distance.

20. The device of claim 19 wherein said distal tip is progressively urged into said remainder of said at least one engagement element, away from said second distance, by said closure element as said closure element moves said delimiting element to said third delimiting position from said second delimiting position.
